(19) United States
(12) Plant Patent Application Publication
(10) Pub. No.: US 2026/0130304 P1
(43) Pub. Date: May 7, 2026

(54) Hop Plant Named 'HBC 1325'

(50) ;

(71) Applicant: HOP BREEDING COMPANY, L.L.C., Yakima, WA (US)

(72) Inventors: Michael D. Ferguson, Yakima, WA (US); Scott Varnum, Selah, WA (US); Jason Perrault, Toppenish, WA (US); Ryan W. Christian, Yakima, WA (US); Jamie Avalos P., Granger, WA (US)

(21) Appl. No.: 18/831,795

(22) Filed: Sep. 10, 2025

Related U.S. Application Data
(60) Provisional application No. 63/732,783, filed on Sep. 18, 2024.

(51) Int. Cl. A01H 6/28; A01H 5/00

(52) U.S. Cl. CPC A01H 6/28; A01H 5/00
USPC PLT/236

(57) ABSTRACT

A new hop plant named ‘HBC 1325’ is disclosed. ‘HBC 1325’ is used for its exceptional and unique aromatic qualities, and is suitable for beer flavoring.

### CROSS-REFERENCE TO RELATED APPLICATIONS

Priority is claimed with respect to U.S. Provisional Patent Application 63/732,783 filed on Sep. 18, 2024.

### STATEMENT REGARDING FEDERALLY SPONSORED RESEARCH OR DEVELOPMENT

None

Genus and species: Humulus lupulus.

Variety denomination: ‘HBC 1325’.

### BACKGROUND OF THE INVENTION

‘HBC 1325’ is a product of a controlled breeding program carried out by the inventors in the Yakima Valley of Washington State (WA). ‘HBC 1325’ was one of several seedlings resulting from a controlled cross made in 2018 with female parent ‘HBC 781’, an unpatented variety, and male parent ‘16-025’ that is a pollen mixture from several unpatented male plants. Seedling plants from this cross were planted in 2019 and screened for disease resistance, vigor, alpha acid content, and sex in field nursery near Granger, WA. Several individual selections were further evaluated in a greenhouse for disease resistance in 2020, and a single plant designated with an internal I.D. number of 18.032.552, was selected in 2020 and asexually reproduced via softwood cuttings, and expanded to 7 plants in an evaluation block near Toppenish, WA. In 2021 the variety was selected for stage 2 yield trials and expanded to a triplicate multi-hill yield trial of 21 plants in an evaluation block near Toppenish, WA. In 2022 the variety was expanded to 150 plants in a stage 3 trial near Toppenish, WA. In 2023, the cultivar was assigned the accession number ‘HBC 1325’ and expanded to 41 acres across multiple test blocks in Washington state, near Toppenish, Granger, Sunnyside, and Wenas, with plants asexually reproduced via softwood cuttings. In 2024, the cultivar was expanded 88 acres across multiple test blocks, again near Toppenish, Granger, Sunnyside, and Wenas, with plants asexually reproduced via softwood cuttings. The ‘HBC 1325’ plants have now been observed and evaluated for several years. Throughout several generations of asexual propagation, ‘HBC 1325’ has been observed to retain its distinctive characteristics and remain true to type.

### BRIEF DESCRIPTION OF THE PHOTOGRAPHS

FIG. 1 illustrates mature bines of a ‘HBC 1325’ hop plant on a trellis at approximately four months of vegetative growth;

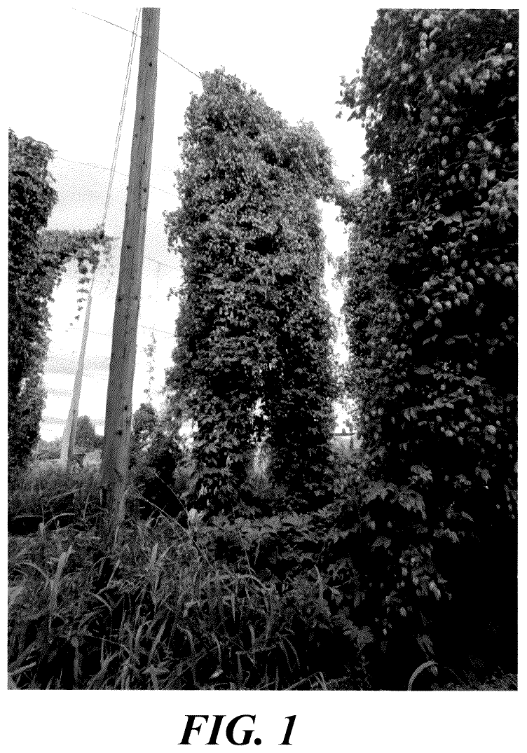

FIG. 2 illustrates mature leaves and cones of a ‘HBC 1325’ hop plant at approximately four months of vegetative growth;

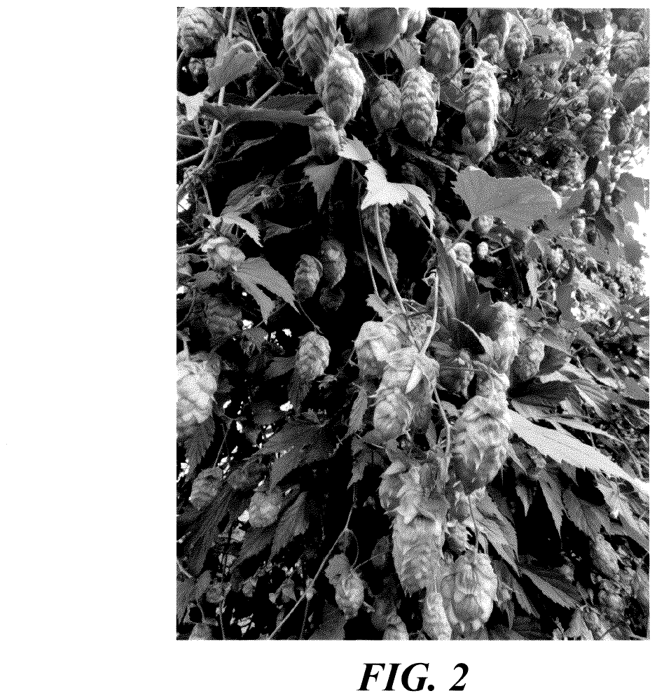

FIG. 3 illustrates a close up of a mature bine of a ‘HBC 1325’ hop plant at approximately four months of vegetative growth;

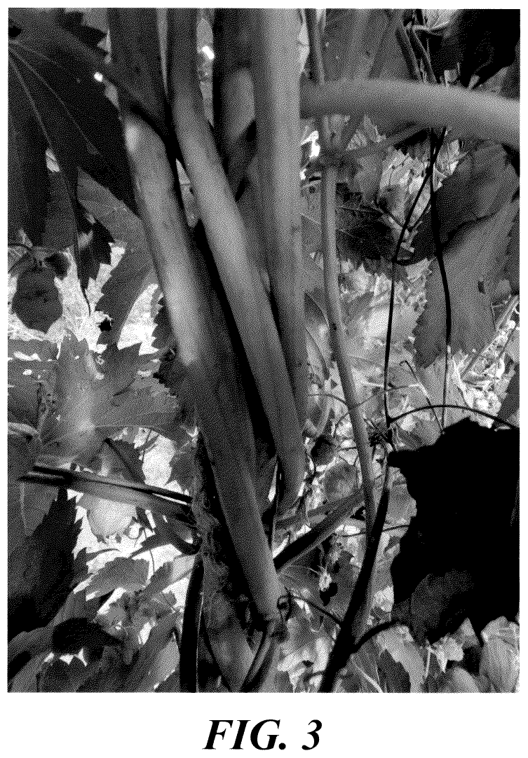

FIG. 4 illustrates a close up of cross-sectioned mature cones of a ‘HBC 1325’ hop plant at approximately four months of vegetative growth; and

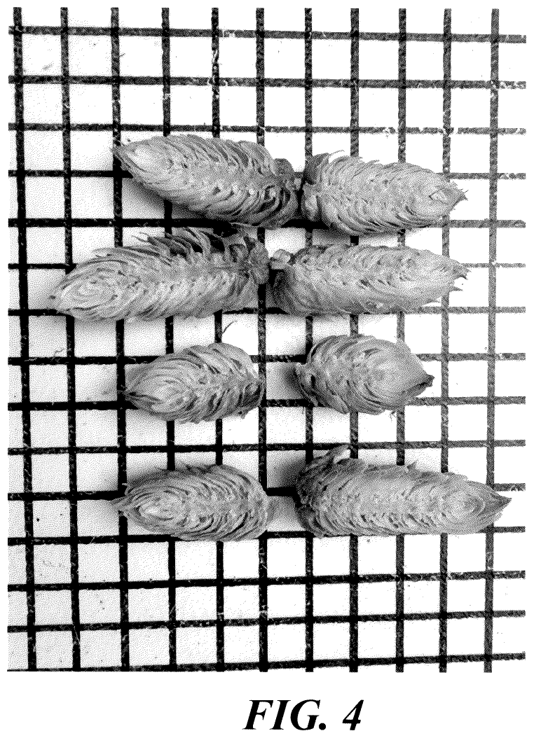

FIG. 5 illustrates a close up of a mature leaf of a ‘HBC 1325’ hop plant at approximately four months of vegetative growth.

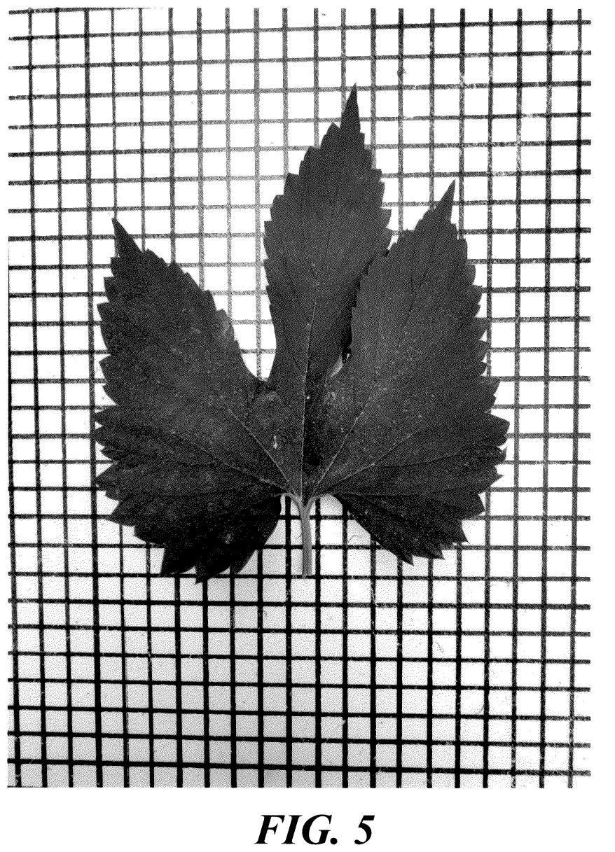

The colors of these illustrations may vary with lighting conditions and, therefore, color characteristics of this new variety should be determined with reference to the observations described herein, rather than from these illustrations at The parent one.

### COMPARISON OF ‘HBC 1325’ TO PARENT PLANT AND COMPARISON CULTIVAR

‘HBC 1325’ though having a distinctive onion and garlic aroma, it has other aroma and flavor characteristics similar to its female parent, the unpatented cultivar ‘HBC 781’. Also, the cones of ‘HBC 1325’ are distinguished from ‘HBC 781’ by typically having a higher alpha acids and a much lower beta acids content.

Table 1. below, sets forth some of the distinguishing characteristics of ‘HBC 1325’ as typically observed, as compared to its female parent ‘HBC 781’, and to the ‘COLUMBUS’ cultivar as a closely comparable cultivar.

TABLE 1

| | | FEMALE | COMPARISON |
|---|---|---|---|
| | INSTANT CV. | PARENT | CV. |
| | ‘HBC 1325’ | ‘HBC 781’ | ‘COLUMBUS’ |
| Alpha | 15.8-20.1 | 10.7-10.9 | 14.5-17.5 |
| (% of cone weight) | | | |
| Beta | 5.2-7.4 | 13.1-13.7 | 4.2-5.9 |
| (% of cone weight) | | | |
| Co-humulone | 16.4-31.7 | 17.4 | 24.9- 30.2 |
| (% of alpha acids) | | | |
| Total Oil (mL/100 g) | 2.3-3.6 | Not available | 2.0-4.0 |
| Aroma Profile | onion and garlic, | berry, fruity | earthy, citrus, |
| | skunk, dank, and | | earthy, woody |
| | herbaceous | | |

### DETAILED BOTANICAL DESCRIPTION

The following description is based on observations made in September of 2023 near Granger, WA. It should be understood that the characteristics described will vary somewhat depending upon cultural practices and climatic conditions, and can vary with location and season. Quantified measurements are expressed as an average of measurements taken from a number of individual plants of the new variety. The measurements of any individual plant or any group of plants, of the new variety may vary from the stated average. Color code designations are by reference to The R.H.S. Colour Chart, 4th ed., Copyright 2001, published by The Royal Horticultural Society of Great Britain.
Ploidy: Diploid.
Sex: Female.
Use: Brewing beer and hop flavored beverages.
Yield: Average yield is expected to be approx. 3,600 pounds per acre.
Plant shape: Columnar.
Growth type: Normal.
Disease and pest resistance or susceptibility:
   Powdery mildew (Podosphaera macularis).—Resistant.
   Downy mildew (Plasmopara halstedii).—Resistant.
Oil analytical characteristics (observed averages):

| | |
|---|---|
| % Alpha Acid (% cone weight) | 17.9 |
| % Beta Acid (% cone weight) | 6.3 |
| % Cohumulone (% alpha acids) | 24.1 |
| Total Oil (ml/100 g of cones): | 2.7 |
| Hop Storage Index (% loss) | 20 to 25 |
| Aroma Profile | Onion and Garlic, Skunky, |
| | Dank, and Herbaceous |

Bine:
   Bine color.—Yellow-Green 146C.
   Stripe present.—Yes.
   Stripe color.—Dark Green-Brown 152D.
   Stipule direction.—Up and down.
   Stipule color.—Yellow-Green 146C.
   Average stipules per bine.—175.
   Typical bine length.—5.8 m.
   Typical internode length on bine.—17 cm to 21 cm.
   Typical internode length on lateral.—53 cm to 132 cm.
   Bine diameter.—14 mm at base; 10.5 mm at nine feet; and 5.0 mm at terminal end of eighteen feet.
Lateral density at middle third of plant: Two Laterals per Node.
   Anthocyanin coloration on main bine.—Medium.
Leaf:
   Arrangement.—Opposite.
   Shape.—Palmately Lobed.
   Number of lobes.—3.
   Apex.—Acuminate.
   Base.—Sagitate.
   Margin.—Serrated.
   Serrations per inch (typically).—5 or 6.
   Pose.—Downward.
   Average length of mature leaf.—12.9 cm.
   Average width of mature leaf.—13.6 cm.
   Leaf blistering on the upper side of leaf blade.—Present.
   Color of mature leaf upper surface.—Yellow-Green 147A.
   Color of mature leaf lower surface.—Green 139A.
   Color of immature leaf upper surface.—Yellow-Green 145A.
   Color of immature leaf lower surface.—Green 147A.
   Petiole color at base.—Yellow-Green 144B.
   Typical petiole diameter (mature).—2 mm to 4 mm.
   Typical petiole length (mature).—6 cm to 11 cm.
   Venation.—Palmate.
   Vein color.—Yellow-Green 144D.
Cone:
   Cone shape.—Oblong.
   Typical cone length.—3.5 cm to 5 cm.
   Typical cone diameter.—1.8 cm to 1.9 cm.
   Average cone weight.—0.86 g.
   Bract shape.—Ovate.
   Bract base shape.—Rounded.
   Bract margin.—Entire.
   Bract tip shape.—Cuspidate.
   Bract tip position.—Alternating.
   Degree of bract opening.—Slightly Open.
   Bract tip color.—Yellow-Green 144A.
   Bract base color.—Yellow-Green 145B.
   Typical bract length.—17 mm to 20 mm.
   Typical bract width.—14 mm to 17 mm.
   Bracteole shape.—Ovate.
   Typical bracteole length.—12 mm to 14 mm.
   Typical bracteole width.—6 mm to 7 mm.
   Bracteole color (upper surface).—Yellow-Green 145C.
   Bracteole color (lower surface).—Yellow-Green 145D.
   Lupulin glands shape.—Pedunculated Oblong Polyps.
   Lupulin glands color.—Yellow-Green 6A.
   Number of lupulin glands per cone.—Varies Greatly (Moderate to Many) with approximately 50,000 lupulin glands per Cone.
   Pickability: Good.
   Time of flowering.—Late July.
   Maturity Date range (early, medium, late).—Late.

1. A new and distinct variety of Hop plant named ‘HBC 1325’, substantially as illustrated and described herein.
